(19) United States
(12) Patent Application Publication
(10) Pub. No.: US 2025/0054606 A1
(43) Pub. Date: Feb. 13, 2025

(54) EMOTIONAL DISORDER EVALUATION SYSTEM, DATA CREATION METHOD, AND SCREENING METHOD

(71) Applicant: UNIVERSITY OF THE RYUKYUS, Okinawa (JP)

(72) Inventor: Shogo ISHIUCHI, Okinawa (JP)

(21) Appl. No.: 18/722,998

(22) PCT Filed: Dec. 21, 2022
(86) PCT No.: PCT/JP2022/047063

§ 371 Date: Jun. 21, 2024

(30) Foreign Application Priority Data
Dec. 24, 2021 (JP) 2021-210944

(51) Int. Cl. G16H 30/40; G16H 20/10; G16H 50/20

(52) U.S. Cl. CPC G16H 30/40; G16H 20/10; G16H 50/20

(57) ABSTRACT

An emotional disorder evaluation system comprising: an acquisition unit configured to acquire image data representing the hippocampus of a subject, and
a specification unit configured to identify volume data representing the volume of the right hippocampus-amygdala transition area of the subject in accordance with the image data.

### TECHNICAL FIELD

The present invention relates to an emotional disorder evaluation system, data creation method, and screening method.

### BACKGROUND ART

The present inventors have conducted research with the aim of establishing diagnostic and therapeutic methods for the following conditions:
1) Diffuse axonal injury caused by traffic accidents, falls, and other incidents,
2) Hypoxic encephalopathy caused by myocardial infarction or stroke,
3) Higher brain dysfunction caused by carbon monoxide poisoning.

As a result, the present inventors have proposed methods and systems for evaluating hippocampal function that do not require large-scale equipment and can accurately and easily assess the hippocampal function of subjects (Patent Documents 1-3).

On the other hand, emotional disorders are mental illnesses with multiple aspects, including biological factors, behavioral factors, social factors, and psychological factors, characterized by changes in mood as the primary clinical symptom.

For example, Patent Document 1 describes a method for determining whether an individual has or is at risk of developing an emotional disorder. The method involves detecting the expression level of connective tissue growth factor (CTGF) in a biological sample derived from the individual. If the expression level of CTGF is increased compared to the control expression level of CTGF, it is determined that the individual has or is at risk of developing an emotional disorder.

### CITATION LIST

### Patent Literature

Patent Literature 1: JP2015-195835
Patent Literature 2: JP2019-63512
Patent Literature 3: JP2019-63513
Patent Literature 4: WO2016-205226

### SUMMARY OF INVENTION

### Technical Problem

However, Patent Document 1 only demonstrates an example where CTGF is upregulated in the amygdala of individuals with major depressive disorder (MDD) compared to controls. This is not sufficient in terms of objectivity and reliability.

The present invention has been made in view of the above circumstances and aims to provide a new emotional disorder evaluation system and a data creation method that can objectively and reliably evaluate emotional disorders. Additionally, the present invention aims to provide a screening method for compounds that have an effect on improving emotional disorders.

### Solution to Problem

In order to solve the above problems, an emotional disorder evaluation system of the present invention is characterized by the following.

An emotional disorder evaluation system comprising:
an acquisition unit configured to acquire image data representing the hippocampus of a subject; and
a specification unit configured to identify volume data representing the volume of the right hippocampus-amygdala transition area of the subject in accordance with the image data.

A method for creating data to evaluate a subject's emotional disorder, comprising:
a step of acquiring image data representing the hippocampus of the subject; and
a step of identifying volume data representing the volume of the right hippocampus-amygdala transition area of the subject in accordance with the image data.

A screening method for compounds with an effect on improving emotional disorders, comprising:
a step of selecting compounds that reduce the volume using volume data representing the volume of the right hippocampus-amygdala transition area.

### Advantageous Effects of Invention

According to the emotional disorder evaluation system and data creation method of the present invention, emotional disorders can be objectively and reliably evaluated. Additionally, the screening method of the present invention enables the screening of compounds that have an effect on improving emotional disorders.

### BRIEF DESCRIPTION OF DRAWINGS

FIG. 1 is a block diagram illustrating the configuration of the emotional disorder evaluation system.

FIG. 2 is a flowchart of the processes executed by the emotional disorder evaluation system.

FIG. 3 is a diagram showing an overview of the volume identification of hippocampal subfields.

FIG. 4 is a diagram showing the BOLD (Blood Oxygen Level Dependent) signal of the bilateral hippocampus-amygdala transition area (HATA) in a subject with right temporal lobe diffuse astrocytoma.

FIG. 5 is a diagram showing the increased volume of the right hippocampus-amygdala transition area (right HATA) in a subject with right temporal lobe diffuse astrocytoma.

FIG. 6 is a diagram showing the results of comparing the volumes of hippocampal and amygdala subfields between women with emotional disorders and healthy controls. It is shown that the volume of the right hippocampus-amygdala transition area (right HATA) is significantly larger in women with emotional disorders.

FIG. 7 is a diagram showing the results of comparing the volumes of hippocampal and amygdala subfields between women with emotional disorders and healthy controls. It is shown that the volume of the right hippocampus-amygdala transition area (right HATA) is significantly larger (P<0.05) in women with emotional disorders.

### DESCRIPTION OF EMBODIMENTS

The present inventor has conducted research on the relationship between emotional disorders and the volume of hippocampal subfields. The present inventor has newly discovered an increase in the volume of the right hippocampus-amygdala transition area (hereinafter sometimes referred to as “right HATA”) in subjects with emotional disorders, leading to the completion of the present invention.

In the present invention, “emotional disorders” include, for example, depression, anxiety, bipolar disorder, postpartum depression, dysthymia, seasonal emotional disorder, schizoaffective disorder, panic disorder, eating disorders, obsessive-compulsive disorder, and post-traumatic stress disorder (PTSD).

The following describes an embodiment of the emotional disorder evaluation system and data creation method of the present invention.

In the present invention, “evaluation of emotional disorders” includes assessing the condition of a subject's emotional disorder (presence or severity), the future risk of developing an emotional disorder, and the effectiveness of treatments.

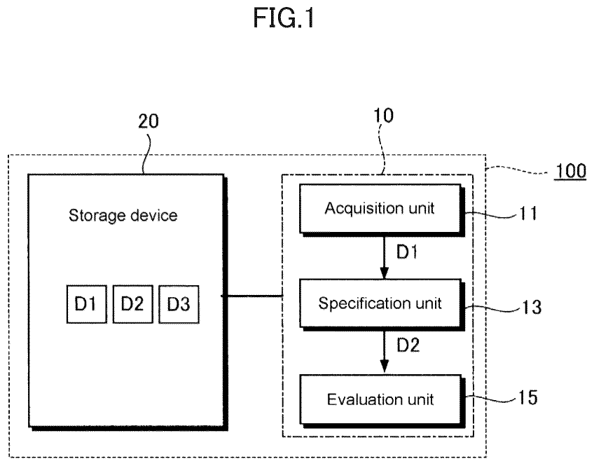

FIG. 1 is a block diagram illustrating the configuration of the emotional disorder evaluation system 100 according to this embodiment. The emotional disorder evaluation system 100 of this embodiment is a system for evaluating the emotional disorders of a subject. Specifically, the emotional disorder evaluation system 100 comprises a control device 10 and a storage device 20.

The control device 10 is one or more processors that control the various components of the emotional disorder evaluation system 100. Specifically, the control device 10 may be composed of one or more processors such as a CPU (Central Processing Unit), SPU (Sound Processing Unit), DSP (Digital Signal Processor), FPGA (Field Programmable Gate Array), or ASIC (Application Specific Integrated Circuit). The control device 10 evaluates the emotional disorder of the subject.

The storage device 20 is one or more memories that store the programs executed by the control device 10 and various data used by the control device 10. The storage device 20 may be composed of known recording media such as magnetic recording media or semiconductor recording media, or a combination of multiple types of recording media. Additionally, a separate storage device 20 (e.g., cloud storage) from the emotional disorder evaluation system 100 may be provided, and the control device 10 may perform writing and reading operations to and from the storage device 20 via a communication network such as a mobile communication network or the internet. That is, the storage device 20 may be omitted from the emotional disorder evaluation system 100.

As illustrated in FIG. 1, the control device 10 in this embodiment realizes multiple functions (an acquisition unit 11, a specification unit 13, and an evaluation unit 15) for evaluating the emotional disorder of a subject by executing programs stored in the storage device 20.

The acquisition unit 11 obtains image data D1 representing the hippocampus and amygdala of the subject from the storage device 20. The image data D1 is displayed on a display device (not shown). The image data D1 is anatomical three-dimensional image data of the hippocampus and amygdala obtained using a device such as a known MRI scanner. When using anatomical three-dimensional image data of the hippocampus, it is preferable to be able to display the images for each hippocampal subfield. Specifically, it is possible to display the tail of the hippocampus, the hippocampal formation, the presubiculum, the parasubiculum, the Cornu Ammonis (CA) 1, CA3, CA4, the granule cell layer of the dentate gyrus (GC-DG), the molecular layer of the hippocampus, the hippocampus-amygdala transition area (HATA), and the alveus. Additionally, it is possible to display the hippocampal fissure.

The image data D1 may be data that has undergone various image processing steps, such as motion correction, skull removal, intensity normalization, white matter separation, 3D tessellation of gray/white matter, and brain surface extraction. Such image processing can be performed using tools like Freesurfer 6.0 (http://surfer.nmr.mgh.harvard.edu).

The specification unit 13 identifies the data representing the volume of the right hippocampus-amygdala transition area (right HATA) of the subject (hereinafter referred to as “volume data” D2). The specification unit 13 of this embodiment identifies the volume data D2 based on the image data D1 acquired by the acquisition unit 11. Any known technique can be used to identify the volume data D2. The volume data D2 may represent the volume itself or it may be data that has undergone predetermined correction processing (for example, a value obtained by multiplying the volume by a coefficient). The identified volume data D2 is stored in the storage device 20.

Furthermore, the size of the head correlates with the size of the body, and the size of the brain correlates with the size of the head. Therefore, it is preferable for the specification unit 13 to identify standardized data of the right hippocampus-amygdala transition area (right HATA) volume as the volume data D2.

As a specific example of a method for identifying the volume data D2, the following methods can be exemplified: 1) analyzing the raw data of hippocampal volume as is, 2) analyzing the value obtained by dividing the hippocampal volume by the volume of the brain or skull, and 3) using the “residual” method to remove shared variations such as brain volume, skull size, or height through multiple regression or ANCOVA. Additionally, adjustments can be made for factors such as gender and age. The specification unit 13 can identify the volume data D2 representing the volume of the hippocampal subfield (right hippocampus-amygdala transition area (right HATA)) in accordance with these methods.

The evaluation unit 15 evaluates the subject's emotional disorder based on the volume data D2 identified by the specification unit 13. Specifically, the evaluation unit 15 compares the volume data D2 identified by the specification unit 13 with reference data representing the volume of the right hippocampus-amygdala transition area (right HATA) to evaluate the state or risk of onset of the subject's emotional disorder. For example, the subject's emotional disorder is evaluated based on the volume data D2 and the data representing the correlation between the reference data and emotional disorders (hereinafter referred to as “preparation data” D3). The preparation data D3 is created in advance and stored in the storage device 20.

The reference data is data representing the volume of the right hippocampus-amygdala transition area (right HATA) that was identified for the subject (or other subjects) in the past. For example, the preparation data D3 is created by associating the reference data with the evaluation of emotional disorders. The evaluation unit 15 identifies the evaluation corresponding to the volume data D2 in the preparation data D3 as the subject's emotional disorder. It should be noted that the evaluation unit 15 may be omitted from the emotional disorder evaluation system.

For example, the evaluation unit 15 can evaluate that the subject does not have an emotional disorder or has a low risk of developing an emotional disorder if the volume of the right hippocampus-amygdala transition area (right HATA) is small. Conversely, the evaluation unit 15 can evaluate that the subject has an emotional disorder or is at risk of developing an emotional disorder if the volume of the right hippocampus-amygdala transition area (right HATA) is large.

In one embodiment of the emotional disorder evaluation system 100, for example, the evaluation unit 15 assesses that the subject has an emotional disorder or is at risk of developing an emotional disorder if the value indicated by the volume data D2 from the specification unit 13 is equal to or greater than a set threshold. Conversely, the evaluation unit 15 assesses that the subject does not have an emotional disorder or has a low risk of developing an emotional disorder if the value indicated by the volume data D2 is less than the set threshold. Such thresholds can be appropriately set based on the correlation between the reference data representing the volume of the right hippocampus-amygdala transition area and emotional disorders. Additionally, thresholds can be set taking into account the characteristics and conditions of the subject.

In another embodiment of the emotional disorder evaluation system 100, for example, the evaluation unit 15 may evaluate the subject's emotional disorder based on the difference between previously measured volume data (A) of the right hippocampus-amygdala transition area and newly measured volume data (B) of the right hippocampus-amygdala transition area. For example, the subject's emotional disorder is evaluated by comparing the difference (absolute value) between volume data (A) and volume data (B) with a predetermined threshold. Such thresholds can be appropriately set based on the correlation between the reference data representing the volume of the right hippocampus-amygdala transition area and emotional disorders. For example, the evaluation unit 15 may assess that the subject's emotional disorder has changed compared to the past if the difference between volume data (A) and volume data (B) is greater than the threshold. Conversely, if the difference between volume data (A) and volume data (B) is smaller than the threshold, the evaluation unit 15 may assess that there is little change in the state of the subject's emotional disorder.

The evaluation unit 15 may assess that the subject's emotional disorder has worsened compared to the past if the volume data (B) is greater than the volume data (A). Conversely, if the volume data (B) is smaller than the volume data (A), the evaluation unit 15 may assess that the subject's emotional disorder has improved compared to the past.

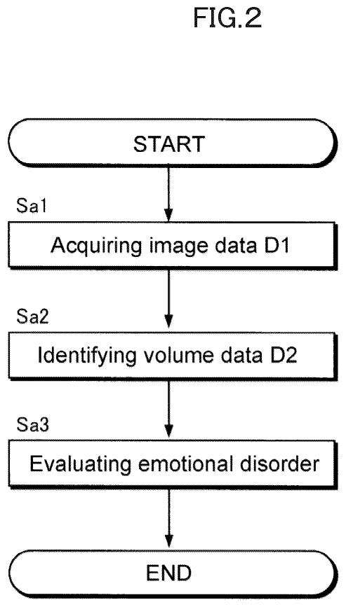

FIG. 2 is a flowchart of the process by which the emotional disorder evaluation system 100 evaluates the emotional disorder of a subject. The process in FIG. 2 is initiated, for example, in response to instructions from the administrator of the emotional disorder evaluation system 100.

The acquisition unit 11 acquires the image data D1 from the storage device 20 (Sa1). The specification unit 13 identifies the volume data D2 representing the volume of the right hippocampus-amygdala transition area of the subject based on the image data D1 acquired by the acquisition unit 11 (Sa2). The evaluation unit 15 evaluates the subject's emotional disorder based on the volume data D2 identified by the specification unit 13 (Sa3). Specifically, the emotional disorder is evaluated based on the correlation between the volume data D2 identified by the specification unit 13 and the emotional disorder.

The emotional disorder evaluation system 100 may also be equipped with, for example, a storage unit that stores information related to the volume of the right hippocampus-amygdala transition area and a display unit that displays the volume of each hippocampal subfield.

The emotional disorder evaluation system 100 of the present invention can be used to evaluate the state (presence or severity) of a subject's emotional disorder or the risk of developing an emotional disorder based on the volume of the right hippocampus-amygdala transition area (right HATA), providing excellent objectivity and reliability.

The present invention is also specified as a method for creating data to evaluate a subject's emotional disorder (hereinafter referred to as “data creation method”).

Specifically, the data creation method includes the steps of acquiring image data D1 representing the subject's hippocampus and identifying volume data D2 representing the volume of the right hippocampus-amygdala transition area of the subject.

Here, as mentioned above, the volume data D2 may represent the volume itself or it may be data that has undergone predetermined correction processing. Data that has undergone predetermined correction processing refers to various forms of data related to the volume of the right hippocampus-amygdala transition area (such as numerical values related to the volume of the right hippocampus-amygdala transition area (right HATA)). For example, using the volume data D2 identified by the data creation method of the present invention, it is possible to evaluate the subject's emotional disorder based on the correlation (preparation data D3) between the reference data representing the volume of the right hippocampus-amygdala transition area (right HATA) and the emotional disorder.

The present invention is also specified as a screening method for compounds with an effect on improving emotional disorders (hereinafter referred to as “screening method”).

Specifically, the screening method of the present invention includes a step of selecting compounds that reduce the volume of the right hippocampus-amygdala transition area using volume data representing this volume.

In the screening method of the present invention, for example, the aforementioned emotional disorder evaluation system and data creation method of the present invention can be used. For example, the screening method of the present invention can include the steps of administering a candidate compound to a subject by a known method, and acquiring volume data representing the volume of the right hippocampus-amygdala transition area (right HATA) before and after administration. Furthermore, the method can include a step of determining that a candidate compound that reduces the volume of the right hippocampus-amygdala transition area after administration has an effect on treating emotional disorders. Compounds that reduce the volume of the right hippocampus-amygdala transition area can be identified as active ingredients in therapeutic agents for emotional disorders.

Such candidate compounds can include, for example, low molecular weight chemical compounds, proteins, polypeptides, peptides, antibodies or their antigen-binding fragments, sugars, polysaccharides, polynucleotides, oligonucleotides, lipids, and so on.

The emotional disorder evaluation system, data creation method, and screening method of the present invention are not limited to the above embodiments.

The present invention is also specified as an evaluation method for assessing a subject's emotional disorder. Specifically, the evaluation method evaluates the subject's emotional disorder based on the volume of the right hippocampus-amygdala transition area. More specifically, for example, the method involves acquiring the aforementioned volume data D2 and comparing it with reference data representing the volume of the right hippocampus-amygdala transition area to evaluate the subject's emotional disorder status or treatment effects.

### EXAMPLE

The following examples are provided to further illustrate the present invention in more detail, but the invention is not limited to these examples.

### Example 1

### <1> Selection of Healthy Subjects

Fifty-eight healthy subjects (mean age 25±4.4 years, range 18-40 years, 31 males with a mean age of 24.5±3.5 years, and 27 females) participated. All subjects were right-handed based on the Edinburgh Handedness Inventory (Oldfield, 1971). Additionally, none of the healthy subjects had any signs or history of neuropsychiatric disorders.

### <2> Behavioral Memory Task

The behavioral memory task was conducted according to the method described in Patent Document 1. One photo at a time was presented on the screen, and the subjects were asked to respond by pressing a button. There were three buttons: one for a new photo they had never seen before (New), one for a photo identical to one seen in previous trials (Same), and one for a photo similar to but different from a previously seen photo (Lure). Subjects were instructed to press the buttons with different fingers for each category (red button for New, blue button for Same, and green button for Lure).

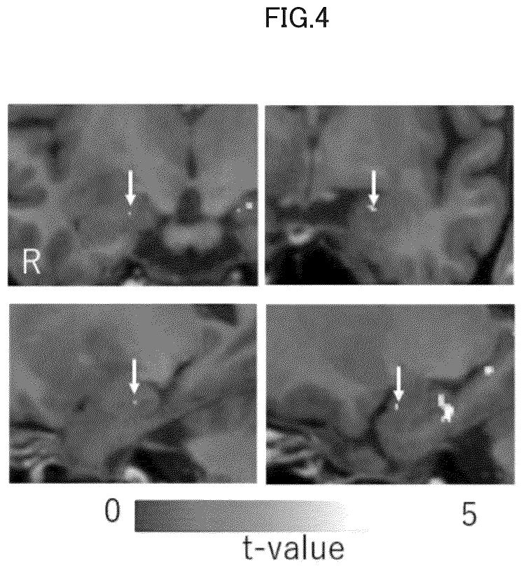

The accuracy rate of the Lure task in this behavioral memory task confirmed that the hippocampal function of both healthy subjects and subjects with right temporal lobe diffuse astrocytoma was preserved. FIG. 4 shows the BOLD (Blood Oxygen Level Dependent) signals in the bilateral hippocampus-amygdala transition areas (HATA) of a subject with right temporal lobe diffuse astrocytoma, indicated by arrows.

### <3> Acquisition of Structural MRI

Anatomical images were obtained using a 3 Tesla MRI scanner (Discovery MR750, GE Medical System, Waukesha, Wisconsin, USA) equipped with a 32-channel head coil and high-order manual shimming for the temporal lobe. Anatomical three-dimensional (3D) T1-weighted images (an example of “image data”) were acquired using a SPGR (spoiled gradient recalled echo) sequence with high-resolution 1 mm slice thickness (matrix size 256×256, field of view 256×256 mm, repetition time 6.9 ms, echo time 3 ms, and flip angle 15 degrees).

### <4> Identifying Volume

All T1-weighted and T2-weighted image data were processed using the freely available software Freesurfer (http://surfer.nmr.mgh.harvard.edu). Volume identification was performed using the fully automated “recon-all” processing pipeline in Freesurfer 6.0, which includes motion correction, skull stripping, intensity normalization, white matter segmentation, 3D tessellation of gray/white matter, and brain surface extraction. Automatic subfield analysis of the hippocampus and amygdala was applied using a probabilistic atlas and a modified version of the Van Leemput algorithm (Van Leemput, et al., 2009). The hippocampus was segmented into subfields for each hemisphere. The hippocampal subfields extracted included the tail of the hippocampus, the subiculum, the presubiculum, the parasubiculum, Cornu Ammonis (CA) 1, CA3, CA4, the granule cell layer of the dentate gyrus (GC-DG), the molecular layer of the hippocampus, the hippocampus-amygdala transition area (HATA), and the alveus.

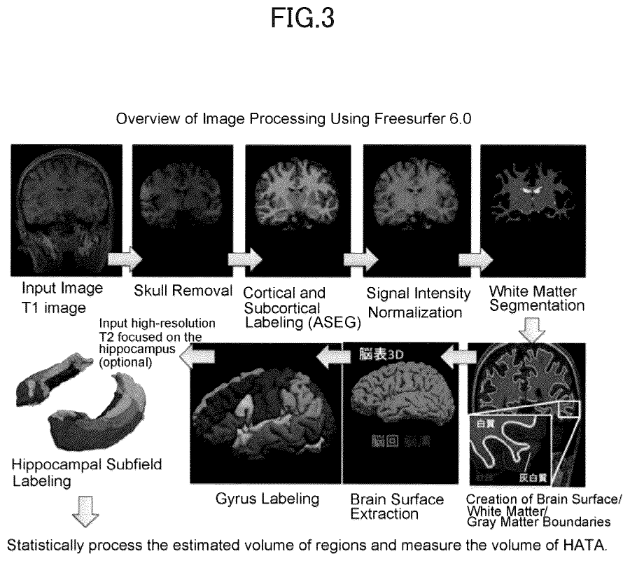

For these reasons, the volume of hippocampal subfields, including the hippocampus-amygdala transition area (HATA), was identified (an example of “volume data”) following the procedure illustrated in FIG. 3, for the purpose of evaluating brain structure alone.

Additionally, the total intracranial volume (TIV) estimated by Freesurfer 6.0 was calculated for brain volume correction.

### <5> Results

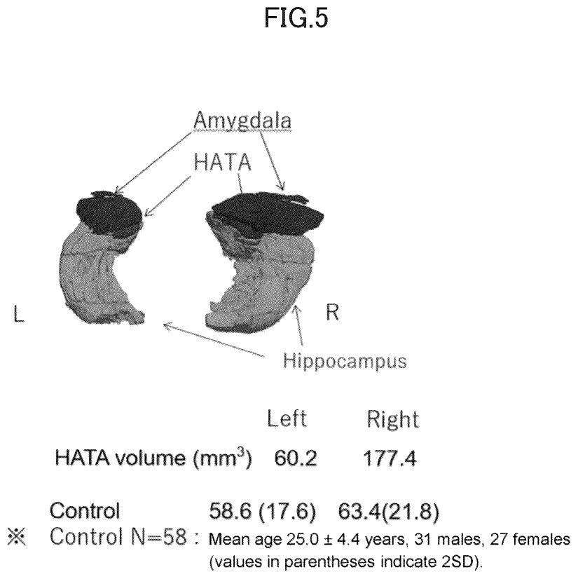

The results are shown in FIG. 5. As indicated in FIG. 5, subjects with right temporal lobe diffuse astrocytoma had a larger volume in the right hippocampus-amygdala transition area (right HATA) compared to healthy subjects, and exhibited emotional disorders such as severe depression.

### Example 2

Twenty-seven healthy female subjects (mean age 25.5±5.2 years, range 18-40 years, right-handed) were selected, and the volumes of the hippocampal and amygdala subfields were identified in the same manner as in Example 1. Similarly, the volumes of the hippocampal and amygdala subfields were identified for females with emotional disorders.

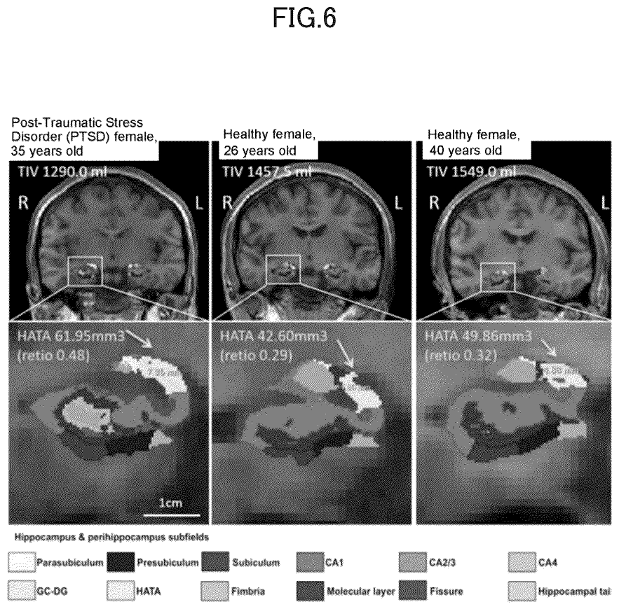

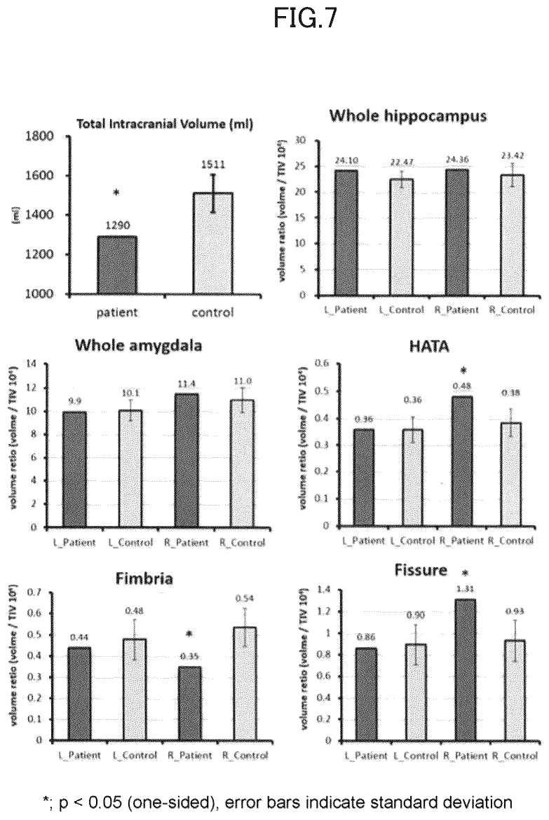

The results are shown in FIGS. 6 and 7, and Table 1.

TABLE 1
Table.Caracteristic of the hippocampus and amygdala subfield retio volumes.

| | Left volume | | | Rignt volume | | |
|---|---|---|---|---|---|---|
| | | Control | | | Control | |
| | Patient | mean volume | | Patient | mean volume | |
| | volume retio | retio.SD | | volume retio | retio.SD | |
| Hippocampus | | | | | | |
| Hippocampal_tail | 4.66 | 3.92 | 0.50 | 4.03 | 3.93 | 0.49 |
| subiculum-body | 1.70 | 1.65 | 0.17 | 1.67 | 1.63 | 0.19 |
| CA1-body | 0.86 | 0.80 | 0.14 | 0.88 | 0.89 | 0.13 |
| subiculum-head | 1.45 | 1.26 | 0.18 | 1.53 | 1.30 | 0.24 |
| hippocampal-fissure | 0.86 | 0.90 | 0.18 | 1.31 | 0.93 | 0.19 |
| presubiculum-head | 0.99 | 0.89 | 0.11 | 0.98 | 0.92 | 0.13 |
| CA1-head | 3.28 | 3.27 | 0.28 | 3.81 | 3.54 | 0.37 |
| presubiculum-body | 1.33 | 1.20 | 0.16 | 1.08 | 1.07 | 0.17 |
| parasubiculum | 0.42 | 0.40 | 0.09 | 0 [symbol] 36 | 0.39 | 0.08 |
| molecular_layer_HP-head | 2.21 | 2.10 | 0.16 | 2.38 | 2.25 | 0.26 |
| molecular_layer_HP-body | 1.64 | 1.51 | 0.15 | 1.64 | 1.58 | 0.16 |
| GC-ML-DG-head | 0.95 | 0.94 | 0.07 | 1.05 | 1.03 | 0.10 |
| CA3-body | 0.57 | 0.54 | 0.08 | 0.68 | 0.63 | 0.10 |
| GC-ML-DG-body | 0.95 | 0.88 | 0.08 | 0.96 | 0.91 | 0.09 |
| CA4-head | 0.79 | 0.77 | 0.06 | 0.85 | 0.85 | 0.09 |
| CA4-body | 0.85 | 0.78 | 0.07 | 0.85 | 0.81 | 0.08 |
| fimbria | 0.44 | 0 [symbol] 48 | 0.09 | 0.35 | 0.54 | 0.09 |
| CA3-head | 0.66 | 0.71 | 0.07 | 0.78 | 0.80 | 0.10 |
| HATA | 0.36 | 0.36 | 0.05 | 0.48 | 0.38 | 0.05 |
| Whole_hippocampal_body | 8.33 | 7.85 | 0.65 | 8.11 | 8.05 | 0.79 |
| Whole_hippocampal_head | 11.11 | 10.70 | 0.81 | 12.22 | 11.44 | 1.25 |
| Whole_hippocampus | 24.10 | 22.47 | 1.60 | 24.36 | 23.42 | 2.29 |
| Amygdala | | | | | | |
| Lateral Nuc | 3.70 | 3.89 | 0.36 | 4.12 | 4.19 | 0.45 |
| Basal Nuc | 2.61 | 2.59 | 0.24 | 3.01 | 2.82 | 0.28 |
| Accessory Basal Nuc | 1.50 | 1.51 | 0.14 | 1.83 | 1.67 | 0.17 |
| Anterior Amygdala Area | 0.30 | 0.32 | 0.04 | 0.36 | 0.37 | 0 [symbol] 05 |
| Central Nuc | 0.22 | 0.23 | 0.03 | 0.28 | 0.26 | 0.04 |
| Medial Nuc | 0.10 | 0.09 | 0.02 | 0.11 | 0.10 | 0.02 |
| Cortical Nuc | 0.14 | 0.14 | 0.02 | 0.16 | 0.15 | 0.02 |
| CTA | 1.06 | 1.03 | 0.12 | 1 [symbol] 23 | 1.12 | 0.11 |
| Paralaminar Nuc | 0.31 | 0.30 | 0.03 | 0.34 | 0.31 | 0.03 |
| whole amygdala | 9.93 | 10.09 | 0.88 | 11.45 | 10.99 | 1.06 |

* The control group consisted of 27 women (mean age 25.5 ± 5.2 years, range 18-40 years, right-handed),
* indicates p < (one-tailed). [symbol]
indicates data missing or illegible when filed

As shown in FIGS. 6 and 7, and Table 1, the total intracranial volume (TIV) of women with emotional disorders was significantly smaller than that of the healthy control group. On the other hand, the volume of the right hippocampus-amygdala transition area (right HATA) in women with emotional disorders was 61.95 mm³, which was significantly higher than the average value of 57.89 mm³ in the healthy control group. When compared as a ratio divided by TIV, it also showed a significantly larger value.

Although not a parenchymal organ, it was confirmed that women with emotional disorders had a significantly larger volume ratio of the right hippocampal fissure (P<0.05) and a significantly smaller volume ratio of the right fimbria (P<0.05) compared to the healthy control group. Therefore, similar to the right hippocampus-amygdala transition area (right HATA), the right hippocampal fissure and the right fimbria can also serve as indicators for evaluating emotional disorders.

As described above, it was confirmed that the volume of the right hippocampus-amygdala transition area of the subject can be identified, and based on this identified volume, the subject's emotional disorder can be evaluated. Additionally, it was confirmed that by using the volume data representing the volume of the right hippocampus-amygdala transition area and using the reduction of this volume as an indicator, compounds with an effect on improving emotional disorders can be screened.

### EXPLANATION OF SYMBOLS

10: Control device
11: Acquisition unit
13: Specification unit
15: Evaluation unit
20: Storage device
100: Emotional disorder evaluation system

1. An emotional disorder evaluation system comprising:
an acquisition unit configured to acquire image data representing the hippocampus of a subject; and
a specification unit configured to identify volume data representing the volume of the right hippocampus-amygdala transition area of the subject in accordance with the image data.

2. The emotional disorder evaluation system according to claim 1,
further comprising an evaluation unit configured to evaluate the emotional disorder of the subject in accordance with the volume data identified by the specification unit and the correlation between the reference data representing the volume of the right hippocampus-amygdala transition area and emotional disorders.

3. The emotional disorder evaluation system according to claim 2,
wherein the evaluation unit evaluates that the subject has an emotional disorder or is at risk of developing an emotional disorder when the value indicated by the volume data is equal to or greater than a set threshold, and evaluates that the subject does not have an emotional disorder or has a low risk of developing an emotional disorder when the value indicated by the volume data is less than the set threshold.

4. The emotional disorder evaluation system according to claim 2,
wherein the evaluation unit evaluates the subject's emotional disorder in accordance with the difference between the previously measured volume data (A) of the subject's right hippocampus-amygdala transition area and the newly measured volume data (B) of the right hippocampus-amygdala transition area.

5. A method for creating data to evaluate a subject's emotional disorder, comprising:
a step of acquiring image data representing the hippocampus of the subject; and
a step of identifying volume data representing the volume of the right hippocampus-amygdala transition area of the subject in accordance with the image data.

6. A screening method for compounds with an effect on improving emotional disorders, comprising:
a step of selecting compounds that reduce the volume using volume data representing the volume of the right hippocampus-amygdala transition area.
